(19) United States
(12) Patent Application Publication
(10) Pub. No.: US 2019/0059818 A1
(43) Pub. Date: Feb. 28, 2019

(54) BALLOON ADVANCEMENT MECHANISM

(71) Applicant: Biosense Webster (Israel) Ltd., Yokneam (IL)

(72) Inventors: Kevin Justin Herrera, West Covina, CA (US); Assaf Govari, Haifa (IL); Christopher Thomas Beeckler, Brea, CA (US)

(21) Appl. No.: 15/689,388

(22) Filed: Aug. 29, 2017

(51) Int. Cl. A61B 5/00

(52) U.S. Cl. CPC A61B 5/6853; A61B 5/6869; A61B 2562/0209; A61B 5/6859

(57) ABSTRACT

A medical instrument includes a shaft, an expandable balloon and a collapsing set of splines. The shaft is configured for insertion into a body of a patient. The expandable balloon is coupled to a distal end of the shaft.

The collapsing set of splines is made at least partially from a shape-memory material having a collapsed pre-formed shape that collapses the balloon.

### FIELD OF THE INVENTION

The present invention relates generally to medical probes, and particularly to design and use of balloon catheters.

### BACKGROUND OF THE INVENTION

Various known catheter designs have expandable distal ends. For example, U.S. Patent application Publication 2015/0223729 describes a system for detecting the dimensions of a cardiac valve annulus. The system includes a compliant balloon and a shaft within the balloon. The catheter may include equally-spaced splines that can expand radially outward such that the electrodes located on the splines may contact the inner wall of the inflated balloon when spline sheath is retracted. The splines may be struts formed from a shape-memory material, such as Nitinol, and heat set in the expanded condition so that the splines self-expand to the expanded condition upon the retraction of spline sheath.

U.S. Patent Application Publication 2015/0025533 describes a medical device that may include a catheter shaft. An expandable balloon may be coupled to the catheter shaft. The balloon may be capable of shifting between a folded configuration and an expanded configuration. A support structure may be coupled to the balloon. The support structure may be capable of shifting the balloon toward the folded configuration.

U.S. Patent Application Publication 2012/0078078 describes a coronary sinus catheter for insertion into a cardiac vessel such as the coronary sinus. The catheter includes a handle and a catheter shaft coupled at one end to the handle. The catheter shaft has a distal end. An anchor is associated with the catheter shaft and is movable between a deployed position and a collapsed position. In the deployed position, the anchor extends radially outward from an outer surface of the catheter shaft for contacting a wall and temporarily anchoring the catheter shaft within the coronary sinus. The catheter also includes an actuator for causing deployment and collapsing of the anchor upon manipulation of the actuator.

A circumferential ablation catheter is described in U.S. Patent Application Publication 2005/0113822. An ablation assembly is mounted at the distal end of the catheter body. The ablation assembly comprises a circumferential ablation element mounted on the distal end of the catheter body, and an inflatable balloon provided in surrounding relation to the circumferential ablation element. The inflatable balloon is adjustable between a radially collapsed position and a radially expanded position. The support member is made of a material having shape-memory, i.e., that can be straightened or bent out of its original shape upon exertion of a mechanical force and is capable of substantially returning to its original shape upon removal of the force.

### SUMMARY OF THE INVENTION

An embodiment of the present invention provides a medical instrument including a shaft, an expandable balloon and a collapsing set of splines. The shaft is configured for insertion into a body of a patient. The expandable balloon is coupled to a distal end of the shaft. The collapsing set of splines is made at least partially from a shape-memory material having a collapsed pre-formed shape that collapses the balloon.

In some embodiments, the medical instrument includes an expanding set of splines, which is made at least partially from a shape-memory material having an expanded pre-formed shape that expands the balloon.

In an embodiment, the expanding set of splines is configured to receive an expanding electrical current via wiring running through the shaft and to expand the balloon in response to the expanding electrical current, and the collapsing set of splines is configured to receive a collapsing electrical current via the wiring and to collapse the balloon in response to the collapsing electrical current.

In another embodiment, a given spline, in the expanding set or in the collapsing set, is configured to be heated by conducting electrical current provided thereto, so as to revert to the expanded pre-formed shape or the collapsed pre-formed shape, respectively.

In another embodiment, the medical instrument includes a heater, which is attached to a given spline in the expanding set or in the collapsing set, and which is configured to be heated by conducting electrical current provided thereto, so as to set the given spline to the expanded pre-formed shape or the collapsed pre-formed shape, respectively.

In another embodiment, the expanding set of splines and the collapsing set of splines are distributed circumferentially around an internal cavity of the balloon.

In some embodiments, the expandable balloon includes a wall containing an internal cavity, and the expanding set of splines and the collapsing set of splines are encapsulated within the wall of the balloon.

In an embodiment, the expanding set of splines and the collapsing set of splines are adhered to an interior or exterior of a wall of the balloon.

In an embodiment, the splines of the expanding set and the splines of the collapsing set are arranged alternately around an internal cavity of the balloon.

In another embodiment, the shape-memory material includes Nitinol.

In an embodiment, the expanding set consists of a first number of splines, and wherein the collapsing set consists of a second number of splines, different from the first number.

There is additionally provided, in accordance with an embodiment of the present invention, a method for manufacturing a medical instrument. The method includes providing an expandable balloon, and coupling to the balloon a collapsing set of splines made at least partially from a shape-memory material having a collapsed pre-formed shape that collapses the balloon. The collapsing set of splines is connected the balloon and to the distal end of a shaft.

There is additionally provided, in accordance with an embodiment of the present invention, a method including inserting into a patient body a medical instrument, which includes a shaft, an expandable balloon coupled to a distal end of the shaft, and a collapsing set of splines made at least partially from a shape-memory material having a collapsed pre-formed shape that collapses the balloon. The balloon is collapsed by setting the collapsing set of splines to the collapsed pre-formed shape.

### BRIEF DESCRIPTION OF THE DRAWINGS

FIG. 1 is a schematic, pictorial illustration of a catheter-based tracking and ablation system, in accordance with an embodiment of the present invention;

FIGS. 2A-2B are schematic views of a balloon assembly of a catheter in an expanded state and in a collapsed state, respectively, in accordance with an embodiment of the present invention; and

FIG. 3 is a flow chart that schematically illustrates a method for expanding and collapsing a balloon catheter, in accordance with an embodiment of the present invention.

### DETAILED DESCRIPTION OF EMBODIMENTS

### Overview

Embodiments of the present invention that are described herein provide improved balloon assemblies for use in medical instruments. In some embodiments, the distal end of a medical instrument, e.g., a catheter, comprises a shaft for insertion into a patient body, an expandable balloon coupled to the distal end of the shaft, and two sets of splines located inside the balloon.

In some embodiments, the balloon assembly has two states—an expanded state and a collapsed state, achieved by operating the two sets of splines located in the balloon, which the physician controls and operates remotely from a console. The catheter is inserted into a patient body in the collapsed state, through a sheath. In the description, hereinafter, one set of splines is named ‘expanding set of splines’ and the other set of splines is named ‘collapsing set of splines’.

In embodiments of the present invention, the splines of the expanding set, as well as the splines of the collapsing set, are made at least partially from a shape-memory material. In the present context, the term “shape-memory material” refers to any material that has a pre-formed shape and returns to its pre-deformed shape when heated.

There are many types of shape-memory materials that are manipulated by temperature, ranging from metal alloys to polymers. The embodiments described herein refer mainly to Shape Memory Alloys (SMA), and more particularly to Nitinol, but the disclosed balloon assemblies can be implemented using any other suitable shape-memory material.

In some embodiments, when no heat is applied, the splines of both sets are not activated and are in their self-accommodating form, and therefore they can bend and take any desired shape of the intended state of the balloon.

In some embodiments, the expanding set of splines has an expanded pre-formed shape that expands the balloon to the expanded state when heated, while at the same time the collapsing set of splines is kept at their self-accommodating state. The collapsing set of splines has a collapsed pre-formed shape that collapses the balloon to the collapsed state when heated, while at the same time the expanding set of splines is kept at its self-accommodating state.

In some embodiments, the two sets of splines are distributed circumferentially around the internal cavity of the balloon in order to assist the uniform expansion and collapse of the balloon. In some embodiments, the two sets of splines are encapsulated within the wall of the balloon. In some embodiments, the two sets of splines are adhered to the interior or exterior of the wall of the balloon.

In some embodiments, the different splines are distributed around the circumference in an alternating fashion in order to balance the splines that expand the balloon into the expanded state and the splines that collapse the balloon back into the collapsed state.

The embodiments described herein refer mainly to implementations having both an expanding set of splines and a collapsing set of splines. In some embodiments, however, the balloon assembly comprises only a collapsing set of splines, and expanding the balloon is carried out using some alternative mechanism. In an example embodiment, the balloon is inflated by pumping pressurized saline into its internal cavity. Before retracting the balloon assembly back into sheath, the saline is pumped out and the collapsing set of splines collapses the balloon to the collapsed state when heated.

The disclosed technique for actively collapsing the balloon assembly into the fully folded state has, for example, the advantage of reducing friction between the collapsed balloon and the sheath, as experienced by the physician. Reducing friction is important both when advancing the balloon assembly via the sheath, and even more so when retracting the balloon assembly back into the sheath. As such, the disclosed techniques enable safer and more reliable advancement and retraction procedures of balloon assemblies.

The disclosed technique for actively expanding the balloon assembly is advantageous, for example, because it may achieve a more controllable and consistent physical contact between the balloon's surface and a target tissue.

### System Description

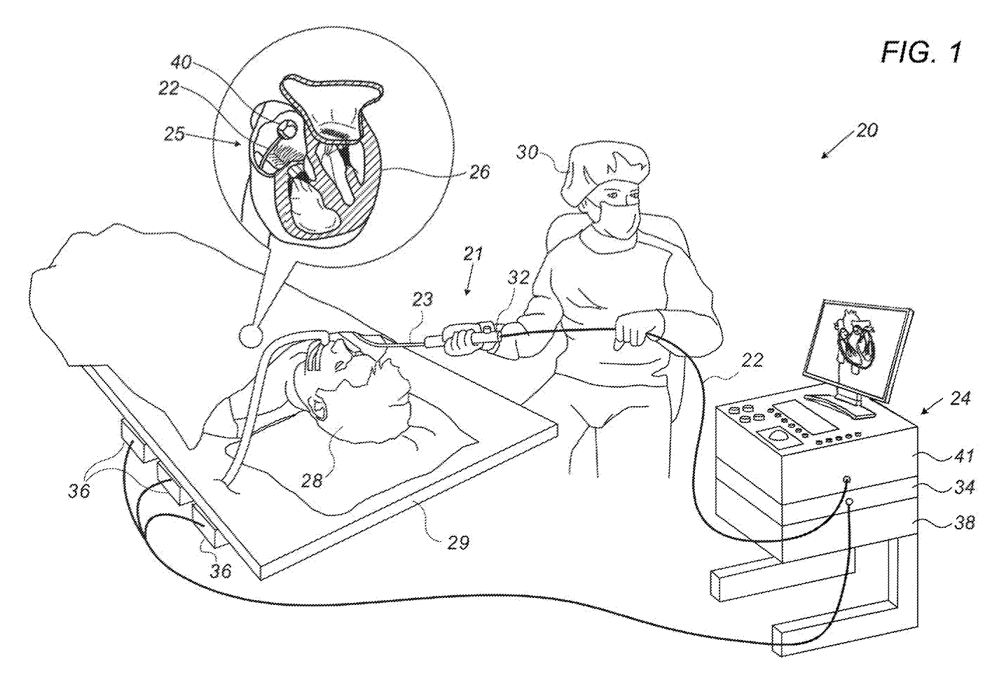

FIG. 1 is a schematic, pictorial illustration of a catheter-based tracking and ablation system 20, in accordance with an embodiment of the present invention. System 20 comprises a catheter 21, where the operator is seen threading the catheter's shaft 22 through a catheter sheath 23. The present example provides a cardiac catheter 21 and a control console 24. In the embodiment described herein, catheter 21 may be used for any suitable therapeutic and/or diagnostic purposes, such as ablation of tissue in a heart 26.

Console 24 comprises a processor 41, typically a general-purpose computer, with suitable front end and interface circuits 38 for receiving signals via catheter's shaft 22 and for controlling the other components of system 20 described herein.

A physician 30 inserts catheter's shaft 22 through the vascular system of a patient 28 lying on a table 29. Catheter 21 comprises a balloon assembly 40 fitted at the distal end of the catheter's shaft 22. During the insertion of catheter's shaft 22, the balloon (not seen) is contained in the sheath 23 in a collapsed position. Balloon assembly 40 is configured to ablate tissue at a target location of heart 26. Physician 30 navigates balloon assembly 40 in the vicinity of the target location in heart 26 by manipulating the catheter's shaft 22 with a manipulator 32 near the proximal end of the catheter as shown in an inset 25. The proximal end of catheter's shaft 22 is connected to interface circuitry in processor 41.

In some embodiments, the position of balloon assembly 40 in the heart cavity is measured by a position sensor (not shown) of a magnetic position tracking system. In this case, console 24 comprises a driver circuit (not shown in the figure), which drives magnetic field generators 36 placed at known positions external to patient 28 lying on table 29, e.g., below the patient's torso. The position sensor is configured to generate position signals in response to sensed external magnetic fields from field generators 36. The position signals are indicative of the position of balloon assembly 40 in the coordinate system of the position tracking system.

This method of position sensing is implemented in various medical applications, for example, in the CARTO™ system, produced by Biosense Webster Inc. (Diamond Bar, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1, whose disclosures are all incorporated herein by reference.

Processor 41 typically comprises a general-purpose computer, which is programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

In some embodiments, console 24 further comprises a current generator 34, which is controlled by processor 41. As will be explained below, current generator 34 is used for expanding and collapsing a distal-end assembly of catheter 21.

### Balloon Advancement Mechanism Using Two Sets of Splines

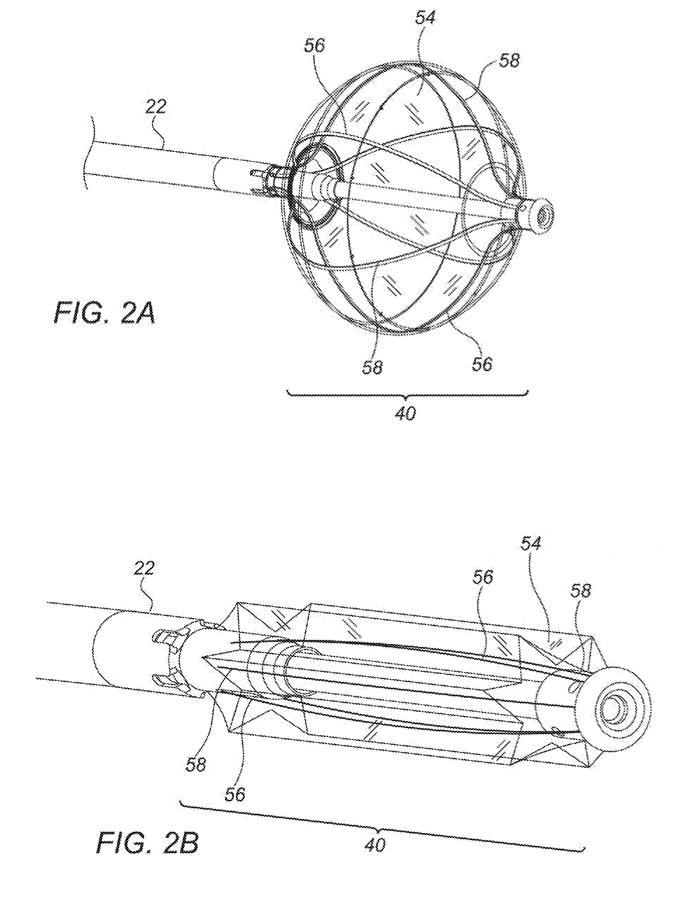

FIG. 2A is a schematic view of balloon assembly 40 in its expanded state, in accordance with an embodiment of the present invention. In some embodiments, assembly 40 comprises an expandable (e.g., inflatable) balloon 54 made from polyethylene terephthalate (PET), polyurethane, polyether block amide, or any other suitable material.

In some embodiments, balloon assembly 40 comprises two sets of splines, an expanding set of splines 56 and a collapsing set of splines 58. The splines are made at least partially from shape-memory material. Splines 56 and splines 58 are typically positioned inside the balloon, and are configured to be heated using electrical current provided via suitable wires that run through the catheter's shaft 22. The physician may operate (e.g., activate and deactivate) each of the two sets of splines independently using console 24.

During the insertion of catheter's shaft 22, balloon assembly 40 is inserted through sheath 23 (shown in FIG. 1 above) in the collapsed position. After being navigated to a target position (e.g., an ostium of a pulmonary vein), in some embodiments, balloon 54 is expanded to an expanded position using splines 56 so as to make physical contact between the external surface of the balloon 54 and tissue at the target location.

The disclosed technique can use other material families of SMA, for example copper-aluminum-nickel. The disclosed technique can also use other types of thermally-responsive materials, such as shape-memory polymers. The shape-memory material may have more than two pre-formed shapes. The description that follows refers to Nitinol as the shape-memory material, by way of example.

SMA typically have two stable phases—the high-temperature phase, called ‘Austenite’ and the low-temperature phase, called ‘Martensite’. Upon heating the SMA to a temperature above its Austenite temperature, the alloy transforms from being a self-accommodated Martensite into an Austenite with a certain pre-formed shape. Upon cooling the SMA to a temperature below its Martensite temperature, the alloy transforms back into its Martensite state.

As splines 56 are heated above their Austenitic temperature, the splines expand into their pre-formed shape and by doing so expand the balloon. Similarly, as splines 58 are heated above their Austenitic temperature, the splines collapse into their pre-formed shape and by doing so collapse the balloon.

In some embodiments, the electrical current passes through the splines themselves, causing the splines to heat due their own electrical resistance. In an alternative embodiment, the electrical current passes through heaters (not shown) attached to the splines.

In some embodiments, as long as the balloon needs to be expanded, Processor 41 inside console 24 maintains the temperature of splines 56 above their pre-formed temperature. When the physician instructs processor 41 to collapse the balloon, the processor lets splines 56 cool below their pre-formed temperature so they become self-accommodating. At this point splines 58 are heated above their pre-formed temperature, collapse into their pre-formed shape and collapse the balloon. Typically, console 24 comprises a suitable input device, e.g., one or more switches or buttons, a keyboard or other interface, for receiving “expand” and “collapse” commands from the physician.

In some embodiments, Splines 56 and splines 58 are distributed circumferentially around the inside of the balloon. In some embodiments, splines 56 and 58 may be assembled in an alternating fashion, e.g., each spline 56 placed between two splines 58, and vice versa. This configuration balances the spines that expand the balloon the splines that collapse and have it back mechanically ready to be easily pulled back into the sheath 23.

In various embodiments, balloon assembly 40 may comprise any suitable number of splines, in any suitable arrangement. For example, the number of splines 56 may be different than the number of splines 58. In some embodiments, balloon assembly 40 may comprise one or more additional splines that are not made of a shape-memory material. In an embodiment, more than two sets of splines are used.

In some embodiments, various electrical and mechanical devices may be disposed on balloon 54 for purposes such as treatment, monitoring, control and diagnostics. Such devices may comprise, for example, ablation electrodes, sensing electrodes, and/or thermocouples.

FIG. 2B is a schematic view of balloon assembly 40 in its collapsed state, in accordance with an embodiment of the present invention. In this state, splines 58 actively collapse and maintain the balloon in its collapsed state and thus secure it in a fully folded configuration.

In some embodiments, the physician uses a user interface on console 24 in order to command the collapse or expansion of the balloon. In response to such a command, processor 41 instructs current generator 34 to heat splines 58 or splines 56. In response, the current generator provides the electrical current to the appropriate set of splines or heating elements associated with the appropriate set of splines.

The example balloon assembly shown in FIGS. 2A and 2B in expanded and collapsed states is chosen purely for the sake of conceptual clarity. In alternative embodiments, the disclosed techniques may use other suitable types, number and arrangement of sets of splines. In example embodiments, the splines may be encapsulated or laminated. In some embodiments, additionally or alternatively to using the expanding set of splines, the balloon may be inflated by pumping pressurized saline into its internal cavity. Moreover, the disclosed techniques are not limited to balloon assemblies, and can be used with other suitable distal-end assemblies.

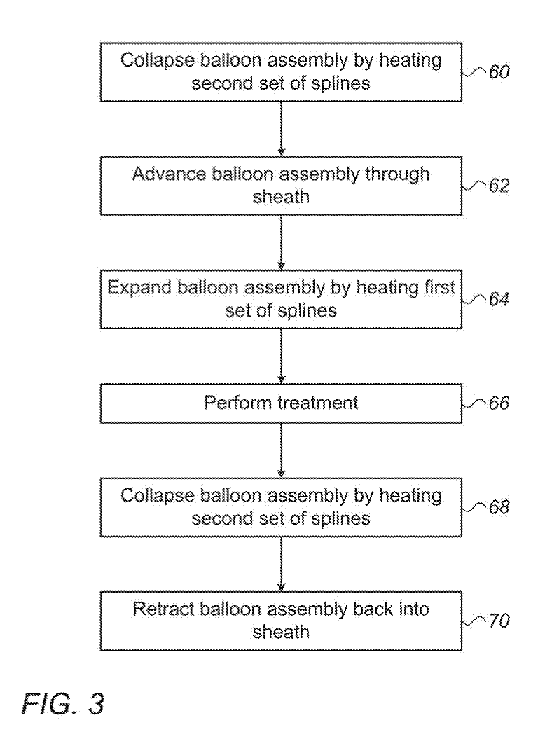

FIG. 3 is a flow chart that schematically illustrates a medical procedure involving expanding and collapsing balloon assembly 40, in accordance with an embodiment of the present invention. The method begins with physician 30 commanding from a user interface at console 24 the collapsing of balloon assembly 40, at a first collapsing step 60.

At an advancement step 62, the physician advances the catheter shaft through the sheath. Advancement continues until the physician decides that the balloon assembly is extracted out of sheath 23 close enough to the target location. At an expansion step 64, the physician expands the balloon assembly by a command from the user interface at console 24.

At a treatment step 66, the physician navigates the expanded balloon in the vicinity of the target location in the heart by manipulating the catheter's shaft 22 with a manipulator 32. The physician then performs the actual treatment, for example an ablation at the target tissue, after which he or she navigates the balloon away from the target location.

At a second collapsing step 68, the physician commands the collapse of balloon assembly 40, this time in order to retract the catheter into the sheath. At a retracting step 70, the physician retracts the balloon assembly back into sheath 23 and out of the patient's body.

The example work-flow presented in FIG. 3 is chosen purely for the sake of conceptual clarity. In alternative embodiments, the disclosed techniques may apply to other work-flow procedures. Moreover, the disclosed techniques are not limited to procedures using balloon assemblies, and can be applied to other procedures using other relevant distal-end assemblies.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A medical instrument, comprising:
a shaft for insertion into a patient body;
an expandable balloon coupled to a distal end of the shaft; and
a collapsing set of splines made at least partially from a shape-memory material having a collapsed pre-formed shape that collapses the balloon.

2. The medical instrument according to claim 1, and comprising an expanding set of splines made at least partially from a shape-memory material having an expanded pre-formed shape that expands the balloon.

3. The medical instrument according to claim 2, wherein the expanding set of splines is configured to receive an expanding electrical current via wiring running through the shaft and to expand the balloon in response to the expanding electrical current, and wherein the collapsing set of splines is configured to receive a collapsing electrical current via the wiring and to collapse the balloon in response to the collapsing electrical current.

4. The medical instrument according to claim 2, wherein a given spline, in the expanding set or in the collapsing set, is configured to be heated by conducting electrical current provided thereto, so as to revert to the expanded pre-formed shape or the collapsed pre-formed shape, respectively.

5. The medical instrument according to claim 2, and comprising a heater, which is attached to a given spline in the expanding set or in the collapsing set, and which is configured to be heated by conducting electrical current provided thereto, so as to set the given spline to the expanded pre-formed shape or the collapsed pre-formed shape, respectively.

6. The medical instrument according to claim 2, wherein the expanding set of splines and the collapsing set of splines are distributed circumferentially around an internal cavity of the balloon.

7. The medical instrument according to claim 2, wherein the expandable balloon comprises a wall containing an internal cavity, and the expanding set of splines and the collapsing set of splines are encapsulated within the wall of the balloon.

8. The medical instrument according to claim 2, wherein the expanding set of splines and the collapsing set of splines are adhered to an interior or exterior of a wall of the balloon.

9. The medical instrument according to claim 2, wherein the splines of the expanding set and the splines of the collapsing set are arranged alternately around an internal cavity of the balloon.

10. The medical instrument according to claim 2, wherein the shape-memory material comprises Nitinol.

11. The medical instrument according to claim 2, wherein the expanding set consists of a first number of splines, and wherein the collapsing set consists of a second number of splines, different from the first number.

12. A method for manufacturing a medical instrument, the method comprising:
providing an expandable balloon;
coupling to the balloon a collapsing set of splines made at least partially from a shape-memory material having a collapsed pre-formed shape that collapses the balloon; and
connecting the balloon and the collapsing set of splines to the distal end of a shaft.

13. The method according to claim 12, and comprising coupling to the balloon an expanding set of splines made at least partially from a shape-memory material having an expanded pre-formed shape that expands the balloon.

14. The method according to claim 13, and comprising inserting in the shaft wiring for providing an expanding electrical current for expanding the expanding set of splines, and a collapsing electrical current for collapsing the collapsing set of splines.

15. The method according to claim 13, and comprising attaching to a given spline, in the expanding set or in the collapsing set, a heater configured to be heated by conducting electrical current provided thereto so as to set the given spline to the expanded pre-formed shape or the collapsed pre-formed shape, respectively.

16. The method according to claim 13, wherein coupling the expanding set and the collapsing set comprises distributing the expanding set of splines and the collapsing set of splines circumferentially around an internal cavity of the balloon.

17. The method according to claim 13, wherein coupling the expanding set and the collapsing set comprises encapsulating the expanding set of splines and the collapsing set of splines within a wall of the balloon.

18. The method according to claim 13, wherein coupling the expanding set and the collapsing set comprises adhering the expanding set of splines and the collapsing set of splines to an interior or exterior of a wall of the balloon.

19. The method according to claim 13, wherein coupling the expanding set and the collapsing set comprises arranging the splines of the expanding set and the splines of the collapsing set alternately around an internal cavity of the balloon.

20. The method according to claim 13, wherein the shape-memory material comprises Nitinol.

21. The method according to claim 13, wherein the expanding set consists of a first number of splines, and wherein the collapsing set consists of a second number of splines, different from the first number.

22. A method, comprising:
inserting into a patient body a medical instrument, which comprises:
a shaft;
an expandable balloon coupled to a distal end of the shaft; and
a collapsing set of splines made at least partially from a shape-memory material having a collapsed pre-formed shape that collapses the balloon; and
collapsing the balloon by setting the collapsing set of splines to the collapsed pre-formed shape.

23. The method according to claim 22, wherein the medical instrument further comprises an expanding set of splines made at least partially from a shape-memory material having an expanded pre-formed shape that expands the balloon, and comprising expanding the balloon by setting the expanding set of splines to the expanded pre-formed shape.
